(19) United States
(12) Patent Application Publication
(10) Pub. No.: US 2023/0404548 A1
(43) Pub. Date: Dec. 21, 2023

(54) Female Urine Specimen Kit

(71) Applicants: Clyde Williams, Scotland Neck, NC (US); Montre Freeman, Roanoke Rapids, NC (US)

(72) Inventors: Clyde Williams, Scotland Neck, NC (US); Montre Freeman, Roanoke Rapids, NC (US)

(21) Appl. No.: 17/867,918

(22) Filed: Jul. 19, 2022

Related U.S. Application Data
(60) Provisional application No. 63/343,702, filed on May 19, 2022.

(51) Int. Cl. A61B 10/00; A61F 5/455

(52) U.S. Cl. CPC A61B 10/007; A61F 5/4556

(57) ABSTRACT

The present invention relates to a female urinary collection device or specimen kit. The kit comprises an adjustable tool designed for a woman to collect a urine sample in a sanitary fashion. The tool has a conformable base component with an opening to allow fluids to travel therethrough, and an accordion like hose connector that transfers the sample from the base component to the urine collection specimen container. The device is flexible and can be used by women of all sizes and of different physical abilities. Further, the device is small enough to be carried in a purse or small carrying bag when not being used as a specimen collection kit.

### CROSS-REFERENCE TO RELATED APPLICATION

The present application claims priority to, and the benefit of, U.S. Provisional Application No. 63/343,702, which was filed on May 19, 2022, and is incorporated herein by reference in its entirety.

### FIELD OF THE INVENTION

The present invention relates to the field of urine specimen collection devices. More specifically, the present invention relates to a device that allows a woman to collect and submit a urine specimen more independently and in a more sanitary way than conventional specimen or sample collection procedures, and that uses a specially configured or shaped receptacle that is attached to a receiving vessel for the specimen. Specifically, the receptacle fits over the urethra and vagina of the user to direct the urine into a collection cup or other receiving vessel. Accordingly, the present disclosure makes specific reference thereto. Nonetheless, it is to be appreciated that aspects of the present invention are also equally applicable to other like applications, devices, and methods of manufacture.

### BACKGROUND

Devices used for the collection of urine specimens from a female are desired in the art because they reduce spillage and avoid assistance from another party, thereby saving the user from embarrassment as well as potential contamination of the specimen during the collection process. One area of concern when collecting a sample is attempting to direct the stream of urine into a small cup that is oftentimes used for the collection of the specimen, such as one being submitted for a drug screening or other medical procedure. A problem with this process is that it can become messy and unsanitary when urine spills onto a user's hands and surrounding area. In addition, the sample can become contaminated if other elements touch the sample while it is being collected requiring the user to submit an additional sample.

Furthermore, the process of submitting urine samples can be incredibly difficult for women with physical limitations and challenges. A process so simple as urine collection may cause women to ask for assistance in directing their urine, which requires them to expose themselves and potentially leading to embarrassment. In addition, such collection procedures may not be a feasible method for women who are visually impaired, or are otherwise unable to easily collect their own urine sample.

Another problem women typically face is the need to excuse themselves while in a public place to use the restroom. Individuals who cannot urinate standing up have no choice but to hold relieving themselves while out in public which can lead to discomfort. Moreover, public restrooms are not always particularly sanitary, making use of the facility undesirable and exposing the individual to other concerns such as bacterial, viral, and other possible contamination.

Specifically, the present invention relates to a urine collection device for women. The device ensures a sanitary process for preventing urine spillage and contamination from external environmental factors. The device features a receptacle which is sized to fit over the urethra and vaginal areas, as well as an adjustable hose connected to the receptacle to direct the urine to the cup or receiving vessel. Furthermore, the device is quite small, economical to produce, and can be carried conveniently in a small bag or purse.

Therefore, there exists a long-felt need in the art for a urine or sample collection device for women to carry with them when they are traveling or away from an area they feel comfortable using the toilet facilities. There is also a long-felt need in the art for a device that prevents the emitted urine from leaking or missing the collection cup. Further, there is a long-felt need in the art for a device that allows women to be capable of collecting urine without needing to seek assistance during a collection procedure. Moreover, there is a long-felt need in the art for a device that allows women to discreetly urinate in an environment where a bathroom is not present or where the sanitary conditions of the toilet are not acceptable for their use. Further, there is a long-felt need in the art for a urine collection device that does not irritate the skin of the user when the receiving receptacle is pressed against the skin of the individual. Finally, there is a long-felt need in the art for a female urine or sample collection device that saves time when collecting a urine sample.

The subject matter disclosed and claimed herein, in one embodiment thereof, comprises a urine or sample collection device for women. The device is a urine funneling system which is pressed against a woman's vaginal area to collect urine expelled from the urethra. The device includes a funnel that collects the urine. The funnel has a first partially ovoid shaped portion that makes up a larger portion of the funnel and a second smaller partially ovoid shaped portion making up a smaller portion which is attached to the larger portion. The funnel is made from a flexible material so that it can conform to the individual. The device further includes an adjustable hose that directs the urine from the funnel component to the receiving vessel or cup. The device size is compatible with a standard urine collection cup, such as those used for drug screening and other medical procedures. Further, the device is compact and can be transported in a purse or small carrying bag.

In a further embodiment, a test strip may be added to the base of the funnel of the receptacle so that the invention can be used as a pregnancy test kit so that the individual does not have to urinate on a stick to collect a sample and thus avoids the mess of conducting a pregnancy test. In an additional embodiment, a wetness sensor may also be provided to detect the presence of a fluid. The sensor and pregnancy test can be connected to a smartphone to allow a user or observer to see if fluid is being collected and if a sample is being provided. The sensor may be a RFID device which may be a UHF, HF or LF sensor.

In this manner, the female urine and specimen collection device of the present invention accomplishes all of the foregoing objects and provides users with a device that prevents spilling or contamination of urine and avoids the necessity of needing to request help when collecting a specimen. The device allows a user to independently collect a urine sample. The device allows for the user to collect urine with one hand and in a comfortable position. The device also allows users with physical limitations to collect urine more efficiently.

### SUMMARY OF THE INVENTION

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a urine or sample collection device for women. The device is a sample collection device which is pressed against a woman's vaginal area to collect urine being expelled from the urethra. The device includes a first ovoid shaped portion forming a larger part and a second ovoid shaped portion forming a smaller portion attached to the larger portion which together form a collection part that collects the urine. The collection part has walls and a base with a hole in the base to allow fluid received in the collection part to pass to a tube or hose which is beneath the base of the collection part and on a side opposite to the surface which receives the fluid. The device includes an adjustable and extendable hose, having first and second ends, with the first end attached to the collection part and covering the opening and the second end connecting to the receiving cup. The hose directs the urine from the collection part to the receiving cup which is provided at another end of the hose. The device is compatible with a standard urine drug test cup. Further, the device is compact and can be transported in a purse or small carrying bag.

The present invention provides a device for efficiently collecting a urine sample from a female. The device is size compatible and can be used by either gender, but it is more specifically designed for use by women. The device is also comprised of a flexible material with the ability to contour to the shape of different users.

In one embodiment, the first and second ovoid shaped portions create a funnel like collection device with an opening in the base of the collection device. The collection piece that surrounds the vaginal area, is made of a flexible material, like a medical grade silicon or other plastic, rubber or silicon material which is flexible. The flexibility of this component would allow the user to shape the funnel into a comfortable shape to fit them as well as to form at least a partial seal around the collection area to prevent contamination from occurring during the collection process. The top or upper edge of the collection part may also have a compressible foam element to assist in creating the seal around the collection area. The invention may be provided in a sanitary bag other seal enclosure in order to prevent the device from being contaminated prior to use.

In another embodiment, the rim of the funnel component is lined with a soft material. The material would be adhered or fused onto the rim and would help prevent irritation of the collection part rubbing on the skin while the device is being used.

In yet another embodiment, the collection component comes in a dark, dull color to prevent people from suspecting anything about the device.

In one embodiment, the hose material is made of an extensible plastic, rubber or silicone or medical grade material that can be compacted or stretched to various sizes. The ability of the hose to stretch to various sizes allows for users to take a urine sample in distinct positions.

In yet another embodiment, the hose component of the device has a length of between 3 inches and 10 inches and a width of between 0.5 and 2 inches. This allows for a steady flow of urine and prevents the hose from backing up and overflowing from the funnel.

In yet another embodiment, the hose is connected to the specimen receiving cup through a hole in the lid of the receiving cup. This would guarantee no spillage as the urine transfers from the hose to the cup. The hose is removably attached to the receiving cup so that it can be removed after the specimen has been collected and then a sealing strip is provided to cover the opening in the specimen receiving cup to prevent contamination until the specimen is tested.

In one embodiment, the urine collection device is made of a washer safe plastic, rubber, or silicone. It is particularly important for anything that encounters human excreta to be able to be washed or disposed of easily in the event that the invention is a single used application.

In yet another embodiment, the collection funnel has two Velcro straps attached to the base. These straps wrap around the user's thighs and secure the funnel to the user. This enables the user to use the device hands free so that the user can focus on holding the urine test cup.

In yet another embodiment, the inside of the receiving component is coated with a hydrophobic material such as, silicone dioxide (SiO2), a wax, or other release type coating. This hydrophobic coating works as a water repellant cover. With the help of gravity, the repellant would allow the urine to slide smoothly into the hose and then to the urine cup.

In one embodiment, the entire collection component has a clip attached to one of its elongated side walls. The hose can be bent upward and put in this clip for more compact storing of the invention when not in use or for shipment.

In another embodiment the base of the collection component is provided with a pregnancy testing strip to allow users to conduct a pregnancy test without the need to collect a urine sample on a stick. In an additional embodiment, a wetness sensor may also be provided to detect the presence of a fluid. The sensor and pregnancy test can be connected to a smartphone to allow a user or observer to see if fluid is being collected and if a sample is being provided. The sensor may be a RFID device which may be a UHF, HF or LF sensor.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the numerous ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the urinary collection device of the present invention in accordance with the disclosed architecture;

FIG. 2 illustrates a perspective view of one potential embodiment of the urinary collection device of the present invention with a top view of the collection component in accordance with the disclosed architecture;

FIG. 3 illustrates a perspective view of one potential embodiment of the urinary collection device from a lateral view with all components constructed in accordance with the disclosed architecture;

FIG. 4 illustrates a perspective view of one potential embodiment of the urinary collection device of the present invention with the hose component connected to the cap of the urine or sample testing cup in accordance with the disclosed architecture; and

FIG. 5 illustrates a perspective view of one embodiment of the urine drug test cup compatible with the urinary collection device presented in FIGS. 1-4 in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth to provide a thorough understanding thereof. It may be evident, however, that innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a urine or sample collection device that specifically provides female users with the ability to independently take a urine-based drug test. There is also a long-felt need in the art for a urinary collection device that allows users to take a drug test while in a plurality of positions. Further, there is a long-felt need in the art for a urinary collection device that is portable and can be stored in a small luggage bag to allow the individual to use the invention when a toilet is not available or if the sanitary condition of the toilet is not acceptable to the individual. Moreover, there is a long-felt need in the art for a device that prevents excessive spillage of urine onto the user's hands, clothes, or surrounding areas. Further, there is a long-felt need in the art for a urinary collection device made of material capable of contouring its shape to fit the user's body and to provide a sanitary and efficient way to conduct a test or collect a sample.

The present invention, in one exemplary embodiment, is a urinary collection or sample collection device. The device has an adjustable funnel for collecting urine from the user and directing it into a standard size urine-based drug test cup or other receptacle if one is needed. Alternatively, the collection component can be used independent of the test cup and/or hose, or just with the hose or tube connected to the collection device to allow some additional directional control to the user. The device comprises a first ovoid shaped portion forming the larger portion and a smaller ovoid shaped portion forming the smaller portion and connected to the larger portion to form the collection component. The collection component has a base and side walls with the side walls sloping to the base to form a funnel like feature. The base has an opening to allow fluid collected in the collection component to flow out of the collection component into a tube or hose connected to the opening. The collection component is pressed against the user's vaginal area and covers the urethra. The device also comprises an accordion style hose component that allows the urine collected in the collection component to reach the urine cup or other receptacle, such as a toilet, without spillage. The hose component can be extended or retracted to any desired size depending on a plurality of scenarios. The hose component is also capable of connecting to the drug test cup through a hole in the container's lid. Further, the device is compact and can be disassembled for easy transportation.

The present invention also includes a method of securing the device upright and in position to be used. The method includes the steps of providing a urinary collection device comprising a shaped funnel base component made of a flexible material for adjustability. The method also includes the step of positioning the assembled device upright while being utilized. Further, the method includes securing the bottom of the hose component to the supplied drug test cup. The hose is fed through or connected to the lid to the test cup or other receptacle. Finally, transporting the device via a carrying bag specifically made for the kit.

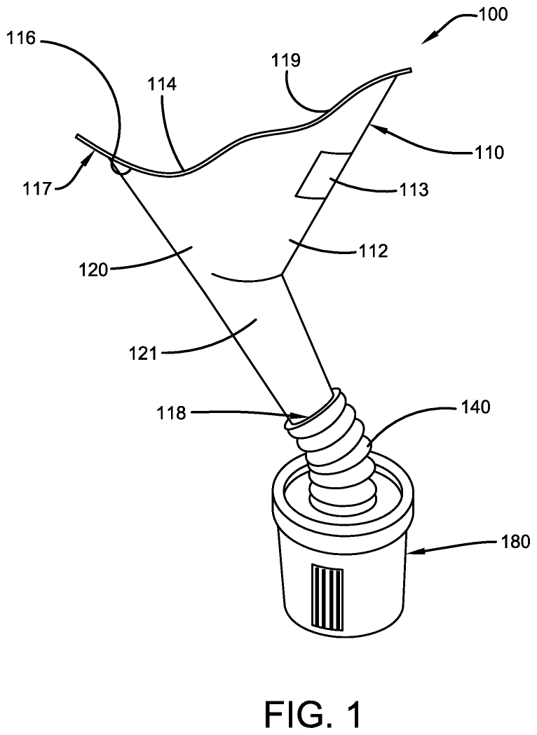

Referring initially to the drawings, FIG. 1 illustrates a perspective view of one embodiment of the urinary collection device 100 of the present invention. In the present embodiment, the urinary collection device 100 is an improved drug testing collection device for females that provides users with a way of easily and independently taking a test without spilling excessive amounts of urine. More specifically, the device 100 comprises a shaped body or collection component 110 with an opening 119 on top to collect urine. The device 100 also includes an accordion-like hose connector 140 which connects from the base or collection component 110 to the urine storage container 180. The collection component 110 bends and contours its shape to securely collect streams of urine and the shape can be easily conformed to different users of the device 100.

The collection component 110 allows for the adjustability of the device 100. Specifically, the device 100 is adjustable and may be used by women with physical limitations. Further, the adjustability is accomplished by providing a base component 110, which is made of a flexible material. The adjustability is also accomplished by a bendable and extendable hose or tube-like connector 140. The device 100 keeps urine contained as it travels from the body or collection component 110, through the tube connector 140, and finally to the urine container 180.

In one embodiment, the body or collector component 110 has a shaped configuration to fit between the legs of the user with an exterior surface 112 and an interior surface 114 made up of a medical grade silicone, plastic, rubber, or silicon material. The body or collector component also has an opening 119 in which the urine travels through. The first opening 116 is much larger and oval or ovoid shaped as it is placed onto the user and catches the urine. The second opening 118 is smaller in diameter, oval or ovoid shaped, and sized to fit inside the connector component 140. Further, the base component 110 has a lip 117 for providing a surface to grip to the user's skin to hold the device in place while being utilized. In another embodiment, the first or top opening or portion 116 is flexible and can be adjusted depending on the needs and/or wants of a user. In another embodiment, the body or collection component 110 can be manufactured into many various sizes and shapes fitting to the user's needs.

In one embodiment, the body or collection component 110 measures 3″ wide, 5″ length, and 6″ tall. This is case of plausible dimensions and many other suitable sizes or shapes as is known in the art, depending on the needs and/or wants of a user can be replicated. Further, the body or collection component 110 is configured in an oval or ovoid shape, but could also be configured in a rectangular, triangular, or circular shape, or any other suitable shape as is known in the art and which could be used to accomplish the present purpose. The base component 110 weighs approximately at least 0.5 lb. and is heavy enough to maintain its shape and not break when washed or used.

In another embodiment, the body or collection component 110 is detachable from the connector hose or tube 140 and the container 180, such that the device 110 can be stored or washed individually if it is not a single use device. Further, the body or collection component lip 117 can be lined with a cloth or gel to prevent irritation. Furthermore, the base component's exterior surface 112 has room for custom indicia, such as the manufacturer of the device. The exterior surface 112 may also be provided with a pocket 113 which has room to store toilet paper or tissues to use once the urinating has been completed. The interior surface 114 may also comprise these capabilities and can be customized to fit a user's needs.

In another embodiment, the body or collection component 110 has an upper reservoir area 120 for extra storage. The reservoir area 120 is oval or ovoid shaped and allows for urine to retain there as it slowly flows through the continuous opening 119 and drains into the connector 140 through the second opening 118. The body component 110 also has a thin nozzle 121 which narrows as it travels towards the second opening 118. This narrowed opening 121 allows the body or collection component 110 to go inside of the hose connector 140 without leaking. This offers a gravity-based flow of the urine and prevents a spill from occurring.

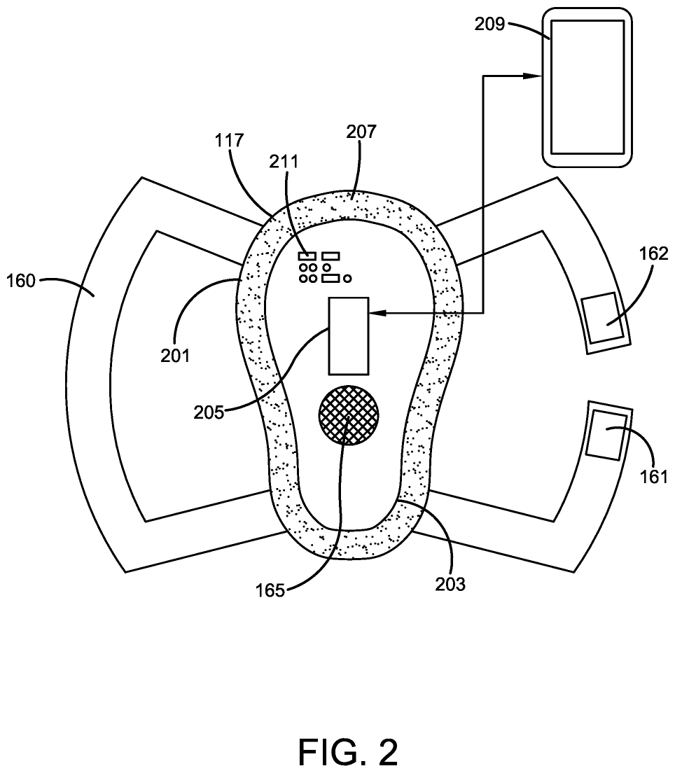

As shown in FIG. 2, the body or collection component 110 comprises a lip surface 117 which is approximately 1 cm (about 0.39 in) in width all around the top of the body or collection component 110. In one embodiment, the lip of the base component 117 can contour to the shape of the user's vaginal area. This lip can be coated with a gel or lined with a material, such as a compressible foam 207, that would prevent irritation of rubber or other material on the skin and to form a seal between the user and the collection component 110 so as to further avoid contamination when giving a drug test.

In another embodiment, the body or collection component 110 has small rectangular shaped indentations 130 for the user's fingers to grip the cup. The finger handles 130 can be any suitable indentation shape as is known in the art and would typically be located on the elongated sides of the exterior wall 112 regarding the base component 110.

Furthermore, the shaped body or collection component 110 can have fine interchangeable filters 165 placed in the thin nozzle compartment 121. These would be finely grated filters for preventing hair or other substance from entering the drug testing container 180. They would be the same diameter as the nozzle component 121, being 0.5 cm (about 0.2 in)-1 cm (about 0.39 in). The filter component 165 can be removed by pushing upward through the second opening 118 or by squeezing the bottom of the body or collection component 110. As previously indicated, the collection component 110 includes a first ovoid portion 201 which forms a larger portion of the collection component 110 and a second ovoid portion 203 which forms a smaller part of the collection component 110. The two portions 201 and 203 have side walls and a base area with an opening to allow urine to flow into the tube or hose that is connected to the opening.

In another embodiment, the shaped collection component 110 comes with a cover 135. This cover lays flush with the rim of the base component 110. The cover 135 can be made from a silicone rubber, Timprene, Neoprene, EPDM, Viton, Butyl or synthetic rubber, plastic, etc., or any other suitable material as is known in the art and maintains the collection component 110 in a sanitary condition if being used for a drug test.

The body or collection component 110 can also include a pregnancy test strip or wetness strip 205. The strip 205 may be attached to a sensor 211 which is connected to a smartphone 209 or other remote device to allow a user or an observer to monitor the test and to see the results of the test. The strip, in addition to pregnancy testing, may be used to see if the user is pouring water or other fluid into the collection component 110 and not urine.

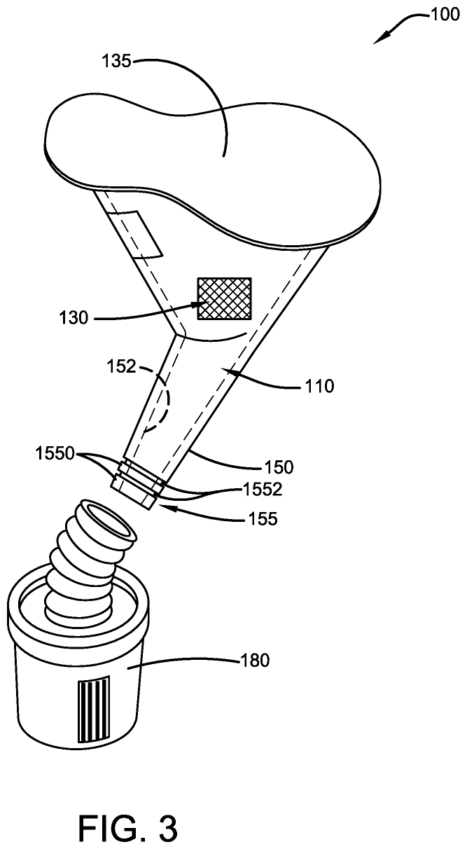

As shown in FIG. 3, the body or collection component 110 has a set of grooves 155 located on the nozzle section 121. These grooves are about 2-3 mm (about 0.12 in) in width and serve as a gripping point for the connector hose 140 to attach to. The grooves 155 are a series of ridges 1550 and valleys 1552. These ridges and valleys work to provide traction to other components and prevent the device from falling apart during use. These grooves are exclusive to the interior surface 114 of the body component 110. The interior of the tube or hose is free from any ridges or valleys as such extrusions would prevent the flow of the urine through the funnel component 110.

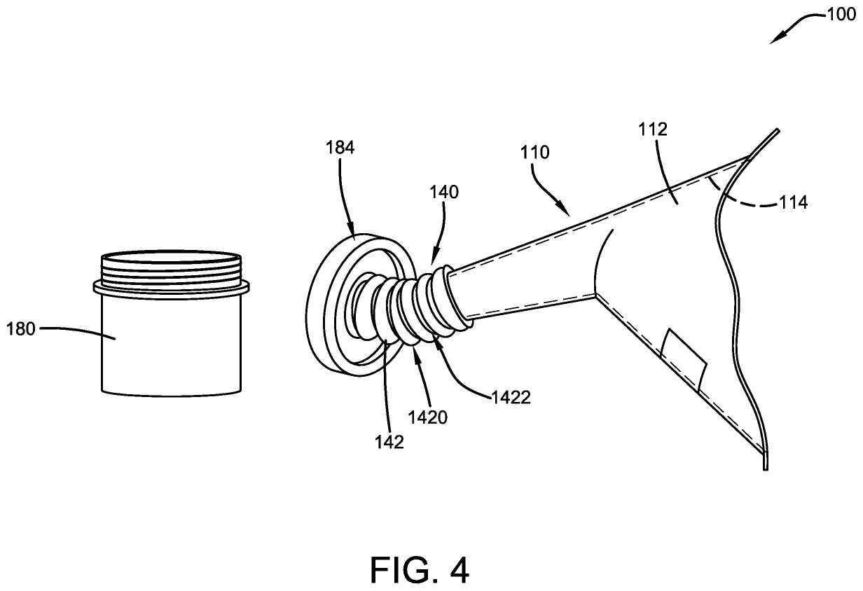

As shown in FIG. 4, the body or collection component 110 is placed inside of the connector component 140. The connector hose is threaded through the adaptive lid 184 which can be placed on the container 180. Thus, the lid 184 has an exterior surface 1940 and an interior surface 1842 that is provided with threads with the ability to be twisted onto the container 180. Specifically, the lid 184 can be slid over the connector 140. The connector hose 140, has an accordion style body 142 and has ridges 1420 and valleys 1422 on the exterior surface of the connector hose 150, as opposed to the interior surface 152. The accordion style hose, with its ridges 1420 and valleys 1422, allows for more compact storage and also provides for grabbing points for the adaptive container lid 184 to be placed. Any other suitable securing feature can be utilized to secure the hose connector 140 in position, as is known in the art, depending on the requirements of a user.

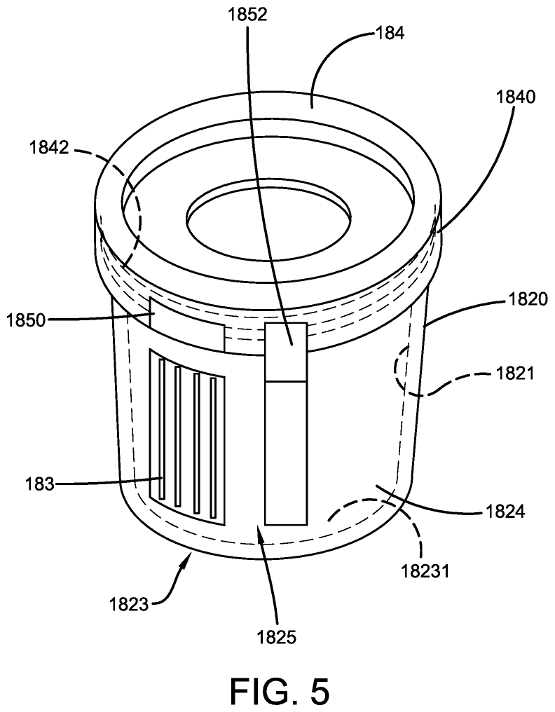

As shown in FIG. 5, the container component 180 has an exterior surface 1820 and an interior surface 1821. Further, the exterior surface 1820 has a continuous back 1824 and extending walls 1825. The container 180 has an exterior bottom material 1823 as well as an interior bottom 18231. On the exterior surface 1820 is a capacity level chart 183 which indicates when the cup is filled to an appropriate level for testing.

In yet another embodiment, the urinary collection body or collection component 110 is manufactured from Silicone rubber, Timprene, Neoprene, EPDM, Viton, Butyl or synthetic rubber, plastic, etc., or any other suitable material as is known in the art.

In yet another embodiment, the urinary collection device 100 has a set of straps 160 for securing the device 100 to a user with possible physical limitation. Straps 160 are wrapped around the base of the base or collection component 110 and are fastened via a system of hook and loop patches on the straps connecting when the hook side 161 latches to the loop side 162. The straps 160 can be customized as is known in the art with possibilities, such as, but not limited to, patterns, logos, emblems, images, symbols, designs, letters, words, characters, animals, advertisements, brands, various combinations thereof.

In use, the urine container 180 has a capacity level strip 183 so that the user can look at the receptacle while using the device to make sure they produce an adequately sized amount to be tested. If the container 180 becomes full, the remaining urine will remain in the hose component 140 which can then be poured out by reversing its previous path of travel through the continuous opening 119.

Still referring to FIG. 5, the container 180 has a set of contamination seals 185, namely a pre-use contamination seal 1850 and a post-use contamination seal 1852. The seal 1850 is placed onto a partial section of the container's exterior surface 1820. The end of the seal stretches to the lid 1840. This seal is broken when the container 180 is ready to be used. The post-use seal 1852 is placed onto the container exterior surface 1820 once a valid urine sample has been collected. The seal is essential to the cup as it prevents users from contaminating the urine specimen or tampering with the test. The seal can be broken when the adaptive lid 184 is removed from the base of the container 180.

Certain terms are used throughout the following description and claims to refer to features or components. As one skilled in the art will appreciate, different users may refer to the same feature or component by different names. This document does not intend to distinguish between components or features that differ in name but not structure or function. As used herein “female urinary collection device,” “Urine collection device,” and “device” are interchangeable and refer to the female urinary collection device 100 of the present invention. This rule of hand also applies for the terms “base component,” “body component,” “Cone-shaped funnel,” and “funnel component” as they are all referring to the main body component 110.

Notwithstanding the foregoing, the urine collection device 100 of the present invention can be of any suitable size and configuration as is known in the art without affecting the overall concept of the invention provided it accomplishes the above-stated objectives. One of ordinary skill in the art will appreciate that the urinary collection device 100 as shown in FIGS. 1-5 are for illustrative purposes only, and many other sizes and shapes of the collection device 100 are well within the scope of the present disclosure. Although the dimensions of the urinary collection device 100 are important design parameters for user convenience, the female urinary collection device 100 may be of any size that ensures optimal performance during use and/or that suits the user's needs and/or preferences.

Various modifications and additions can be made to the exemplary embodiments discussed without departing from the scope of the present invention. While the embodiments described above refer to features, the scope of this invention also includes embodiments having different combinations of features and embodiments that do not include all of the described features. Accordingly, the scope of the present invention is intended to embrace all such alternatives, modifications, and variations fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matters are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner like the term “comprising” as “comprising” is interpreted when employed as a transitional word in a claim.

1. A sample collection device comprising:
a collection body comprised of a side wall, a base, a rim extending around the collection body, and a pocket disposed on an exterior surface of the collection body, wherein the side wall slopes to the base to form a funnel and the base comprises an opening;
a hose comprised of a first and a second end, wherein the opening is connected to the first end of the hose and the hose is both flexible and extendable; and
a container provided to collect a sample and having a lid with an opening therein, wherein the second end of the hose is connected to the container, and further wherein the collection body is conformable to fit an anatomy of a female user; and
wherein the rim is provided with a gel to form a seal between the collection body and the user.

2. The sample collection device as recited in claim 1, wherein the collection body further comprises a first ovoid shaped portion and a second ovoid shaped portion connected to the first ovoid shaped portion.

3. The sample collection device as recited in claim 2, wherein the first ovoid shaped portion is larger than the second ovoid shaped portion.

4. (canceled)

5. (canceled)

6. The sample collection device as recited in claim 1, wherein the collection body is comprised of a width of three inches, a length of five inches and a height of six inches.

7. The sample collection device as recited in claim 1, wherein the collection body is comprised of a medical grade silicone, a rubber, a silicone or a plastic.

8. The sample collection device as recited in claim 1 further comprising a filter positioned in the opening of the collection body.

9. The sample collection device as recited in claim 1 further comprising a test strip positioned on the base of the collection body.

10. The sample collection device as recited in claim 9, wherein the test strip is connected to a sensor.

11. The sample collection device as recited in claim 10, wherein the sensor is in wireless communication with a smartphone.

12. The sample collection device as recited in claim 9, wherein the test strip detects at least one of a wetness, a urine or a pregnancy.

13. A female urination device comprising:
a collection body conformable to an anatomy of a female user, the collection body comprised of a first ovoid shaped portion, a second ovoid shaped portion, a wall that slopes toward a base having a hole, and a plurality of finger handles disposed on an exterior surface of the collection body;
a flexible tube having a first end and a second end, wherein the first end is removably attached to the base around the hole, and the second end is removably attached around an opening in a lid;
a receptacle for receiving a sample from the female user, wherein the receptacle is removably attached to the lid by a threaded closure and comprises a capacity level indicator disposed on an exterior surface of the receptacle; and
a set of contaminant seals positioned at least partially on the lid.

14. The female urination device as recited in claim 13, wherein the collection body is comprised of a medical grade silicone, a rubber, a silicone or a plastic.

15. The female urination device as recited in claim 13, wherein the first ovoid shaped portion is larger than the second ovoid shaped portion.

16. The female urination device as recited in claim 15 further comprising a test strip positioned along the collection body.

17. The female urination device as recited in claim 16, wherein the test strip is capable of detecting a urine, a wetness and a pregnancy.

18. The female urination device as recited in claim 17, wherein the test strip is connected to a sensor and the sensor is in wireless communication with a smartphone.

19. The female urination device as recited in claim 13 further comprising an interchangeable filter.

20. A specimen kit for use by a woman, the specimen kit comprising:
a collection body;
a removable grated filter positioned within the collection body;
a container for receiving a specimen; and
a tube connecting the collection body to the container;
wherein the tube is flexible and extendable and comprises a first end connected to the collection body and a second end connected to a lid having an opening, wherein the lid is removably connected to the container; and
further wherein the collection body comprises a first portion, a second portion that is smaller in size than the first portion, a side wall that slopes towards a base having an opening therein that is in fluid communication with the tube, a removable cover, and a plurality of finger handles disposed on an exterior surface of the collection body.
